(19) United States
(12) Patent Application Publication
(10) Pub. No.: US 2010/0241157 A1
(43) Pub. Date: Sep. 23, 2010

(54) FILTER AND WIRE WITH DISTAL ISOLATION

(75) Inventors: Kevin Brounstein, Fremont, CA (US); Bradley White, Palo Alto, CA (US); Brendon Carver, Belmont, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/793,878

(22) Filed: Jun. 4, 2010

Related U.S. Application Data
(63) Continuation of application No. 11/278,503, filed on Apr. 3, 2006.

(51) Int. Cl. A61M 29/00

(52) U.S. Cl. 606/200

(57) ABSTRACT

A wire may be configured to provide distal isolation from proximal movement, and a filter may be deployed at or near a distal portion of the wire. A filter may be deployed on a spinner tube that is rotatably disposed about a distal portion of the wire. Axial movement of the filter wire may be at least partially absorbed by the spinner tube. A telescoping wire may include a solid proximal wire section, a solid distal wire section and a joining section that is disposed between the solid proximal wire section and the solid distal wire section. The joining section may translate rotational movement between the distal and proximal sections.

### RELATED APPLICATIONS

This application is a continuation of U.S. application Ser. No. 11/278,503 filed Apr. 3, 2006.

### TECHNICAL FIELD

The present invention pertains generally to wires such as filter wires and pertains more particularly to wires such as filter wires that provide distal isolation from proximal movement.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences because the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PICA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

A wide variety of medical devices have been developed for medical use, for example, intravascular use. Of the known devices, each has certain advantages and disadvantages. There is an ongoing need to provide alternative devices. In particular, there is an ongoing need for wires such as filter wires that provide distal isolation from proximal movement.

### SUMMARY

The invention pertains generally to wires such as filter wires and pertains more particularly to wires such as filter wires that provide distal isolation from proximal movement.

Accordingly, an illustrative but non-limiting example of the invention may be found in a filter assembly that includes a filter wire and a spinner tube that is rotatably disposed about a distal portion of the filter wire. A filter may be secured to the spinner tube. In some instances, axial movement of the filter wire may be at least partially absorbed by the spinner tube.

Another illustrative but non-limiting example of the invention may be found in a filter assembly that includes a filter wire and a spinner tube rotatably disposed about a distal portion of the filter wire. A filter may be secured to the spinner tube, and a spinner tube stop may be secured to the filter wire such that the spinner tube is disposed distal of the spinner tube stop. An adaptable segment may be disposed between the spinner tube stop and the spinner tube proximal end.

Another illustrative but non-limiting example of the invention may be found in a telescoping wire that includes a solid proximal wire section, a solid distal wire section and a joining section that is disposed between the solid proximal wire section and the solid distal wire section. The joining section may be configured to translate rotational movement between the proximal section and the distal section yet permit the distal section to move axially relative to the proximal section.

The above summary of the present invention is not intended to describe each disclosed embodiment or every implementation of the present invention. The Figures, Detailed Description and Examples which follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a partial cross-sectional view of a filter assembly in accordance with an illustrative but non-limiting example of the invention;

FIG. 2 is a partial cross-sectional view of a filter assembly in accordance with an illustrative but non-limiting example of the invention;

FIG. 3 is a partial cross-sectional view of a filter assembly in accordance with an illustrative but non-limiting example of the invention;

FIG. 4 is a partial cross-sectional view of a filter assembly in accordance with an illustrative but non-limiting example of the invention;

FIG. 5 is a partial cross-sectional view of a filter assembly in accordance with an illustrative but non-limiting example of the invention;

FIG. 6 is a side view of a telescoping wire in accordance with an illustrative but non-limiting example of the invention;

FIG. 7 is a side view of a telescoping wire including an embolic filter in accordance with an illustrative but non-limiting example of the invention;

FIG. 8 is an exploded partial cross-sectional view of a telescoping wire in accordance with an illustrative but non-limiting example of the invention;

FIG. 9 is a cross-section taken along line 9-9 of FIG. 8;

FIG. 10 is a partial cross-sectional view of a distal portion of a telescoping wire in accordance with an illustrative but non-limiting example of the invention; and

FIG. 11 is a diagrammatic cross-section of a proximal portion of a telescoping wire adapted to interact with the distal portion of FIG. 12.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

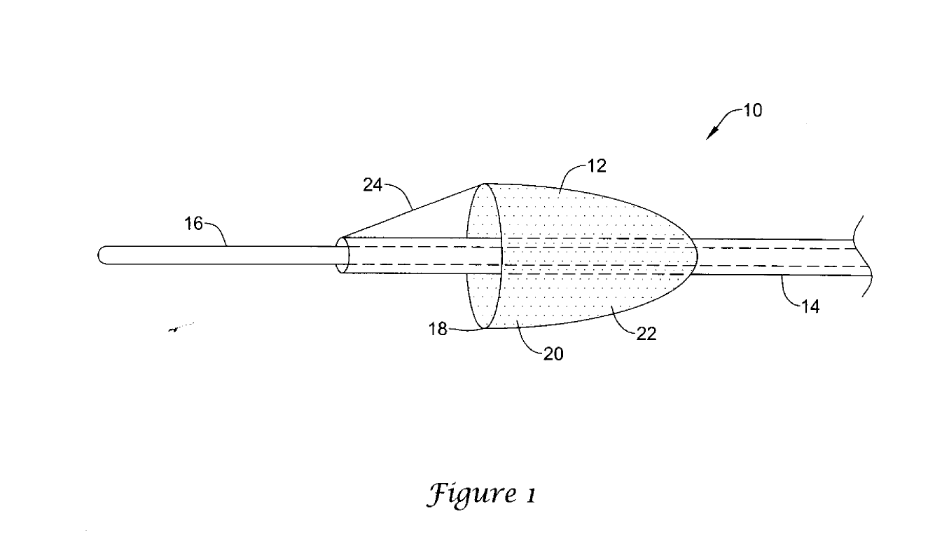

FIG. 1 is a partial cross-sectional view of an illustrative but non-limiting filter assembly 10. The filter assembly 10 includes a filter 12 that is disposed on a spinner tube 14. The spinner tube 14 is disposed about an elongate shaft or wire 16. Wire 16 may be a guidewire, a filter wire or some other solid or non-solid elongate shaft. In some instances, spinner tube 14 may be rotatably and/or slidably disposed about wire 16. Spinner tube 14 may be formed of any suitable metallic or polymeric material. Wire 16 may be formed of any suitable metallic material. In some cases, wire 16 may be formed of a polymeric material possessing desired strength characteristics, such as, perhaps, carbon fiber, liquid crystal polymer, or even a fiber-reinforced polymer or polymer blend.

Filter 12 may, as illustrated, include a filter loop 18 and a filter membrane or fabric 20 that is secured to filter loop 18. In some cases, if desired, filter membrane 20 may be drilled (for example, formed by known laser techniques) or otherwise manufactured to include a plurality of openings 22. These holes or openings 22 can be sized to allow blood flow therethrough but restrict flow of debris or emboli floating in the body lumen or cavity.

In general, filter 12 may be adapted to operate between a first generally collapsed configuration and a second generally expanded configuration (as shown in FIG. 1, for example) for collecting debris in a body lumen. To this end, in at least some embodiments, loop 18 may be formed of or may include a “self-expanding” shape-memory material such as nickel-titanium alloy, which is capable of biasing filter 12 toward being in the second expanded configuration. Additionally, filter loop 18 may include a radiopaque material or include, for example, a radiopaque wire disposed about a portion thereof. Some further details regarding these and other suitable materials are provided below.

One or more struts 24 may extend between filter loop 18 and spinner tube 14. Strut 24 may be secured at one end to the filter loop 18 and at the other end to the spinner tube 14. Strut 24 may be secured at either end in any suitable manner, including soldering, laser welding, adhesives, or mechanically such as by wrapping one end of strut 24 several times about filter loop 18 and wrapping the other end of strut 24 several times about spinner tube 14. It will be recognized the configuration and even the number of struts 24 may be tailored and/or customized for a particular intervention.

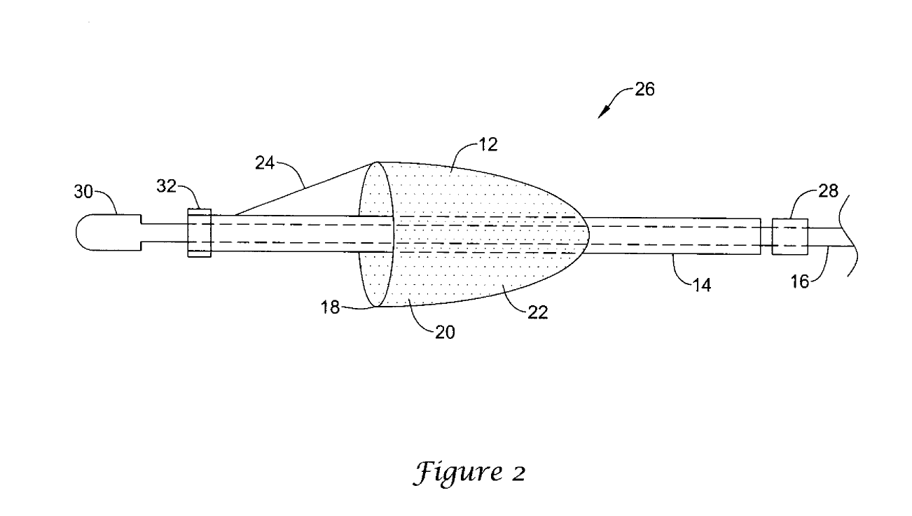

It can be appreciated that spinner tube 14, as shown in FIG. 1, may be free to both rotate and translate relative to wire 16. In some instances, there may be advantages to at least partially limiting axial movement of spinner tube 14 with respect to wire 16. FIG. 2 provides an illustrative but non-limiting example of a filter assembly 26 that includes structure that may limit some axial movement.

Filter assembly 26 is similar to filter assembly 10 in that both include similar elements denoted by similar reference numbers. In FIG. 2, it can be seen that filter assembly 26 includes a spinner tube stop 28. Spinner tube stop 28 may be secured to wire 16 and may serve to provide a proximal limit to relative movement of spinner tube 14 relative to wire 16. Spinner tube stop 28 may be formed of any suitable material, having any suitable geometry, and may be secured to wire 16 using any suitable technique.

Spinner tube stop 28 may, for example, be a short cylindrical length having an inner diameter suitable to fit over wire 16, and an outer diameter sufficient to impede axial movement of spinner tube 14. If, for example, spinner tube stop 28 is polymeric, it may be adhesively secured to wire 16. If, however, spinner tube stop 28 is metallic, it may be soldered, laser welded, or sonically welded.

In FIG. 2, wire 16 includes a floppy tip 30. Floppy tip 30 may be formed of any suitable material, having any suitable geometry, and may be secured to wire 16 using any suitable technique. Floppy tip 30 may be integrally formed with wire 16, or floppy tip 30 may be separately formed and then subsequently secured to wire 16. Floppy tip 30 may be formed of the same material as wire 16, or from a different material.

Spinner tube 14 may, as illustrated in FIG. 2, include a distal stop 32. Distal stop 32 may be secured to spinner tube 14. In some instances, distal stop 32 may cooperate with floppy tip 30 to limit distal travel of spinner tube 14. Floppy tip 30 may be formed having an outer diameter that is sufficient to contact at least one of spinner tube 14 and distal stop 32. Distal stop 32 may be formed of any suitable material, having any suitable geometry, and may be secured to spinner tube 14 in any suitable manner. Distal stop 32 may be integrally formed with spinner tube 14, or may be separately formed and then subsequently secured to spinner tube 14.

It will be recognized that spinner tube 14 has a length, and that spinner tube stop 28 may be secured to wire 16 at any desired location. In some cases, spinner tube stop 28 may be secured to wire 16 at a distance from floppy tip 30 that is just greater than the length of spinner tube 14, thereby permitting only limited relative axial movement between spinner tube 14 (and hence filter 12) and wire 16.

In some instances, greater movement may be desired, and in such cases spinner tube stop 28 may be secured to wire 16 at a distance that is greater or even much greater than a length of spinner tube 14. It is contemplated that the relative distance between spinner tube stop 28 and floppy tip 30 may be from about 1 millimeter to about 20 millimeters longer than a length of spinner tube 14.

It will be recognized that other medical devices such as therapeutic catheters, diagnostic catheters, guide catheters, stent delivery catheters, endoscopic devices, laparoscopic devices, and others may be advanced over wire 16. This may cause wire 16 to move, thereby transmitting axial movement to filter 12.

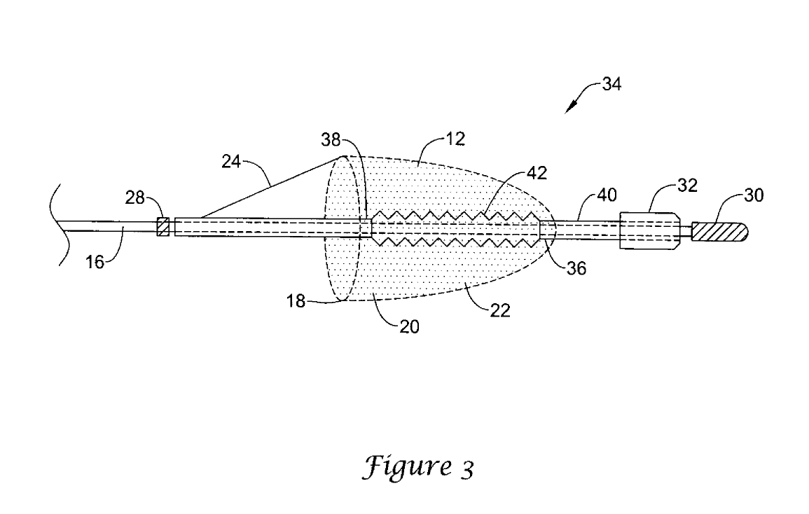

Movement of filter 12, once positioned, may be detrimental. Therefore, in some cases, it may be desirable to dampen relative movement of filter 12. FIGS. 3, 4 and 5 provide illustrative but non-limiting examples of filter assemblies that are configured to dampen relative movement of filter 12. These filter assemblies are configured such that axial movement of wire 16, relative to filter 12, is at least partially attenuated.

FIG. 3 illustrates a filter assembly 34 that shares a number of components with filter assembly 26 (FIG. 2). In this Figure, filter 12 is shown in phantom to better illustrate internal elements of filter assembly 34. Filter assembly 34, however, introduces a spinner tube 36 that is somewhat different. Spinner tube 36 includes a proximal portion 38, a distal portion 40 and a compliant portion 42 that is disposed between the proximal portion 38 and the distal portion 40. The compliant portion 42 is configured such that it can change its length in response to axial movement of wire 16.

In some instances, compliant portion 42 may be considered as having a spring force constant. This means that compliant portion 42 may exert a force counter to any force being applied to it. For example, if wire 16 is being pushed distally, and spinner tube 36 has contacted spinner tube stop 28, compliant portion 42 may exert a force countering the distal force being applied by wire 16. Compliant portion 42 may compress, or shorten in length, in response to wire 16 being pushed distally. Conversely, if wire 16 is being pulled proximally, and distal stop 32 has contacted floppy tip 30, compliant portion 42 may elongate, or increase in length, in response to wire 16 being pulled proximally.

Spinner tube 36, or at least compliant portion 42, may be formed of any suitable material that can provide these properties. In some cases, compliant portion 42 may include an accordion section of spinner tube 36. An accordion section may be able to change its length in response to an applied force, thereby attenuating the impact of that applied force. While not expressly illustrated, compliant portion 42 may have a cylindrical configuration, and may be formed of an elastomeric polymer.

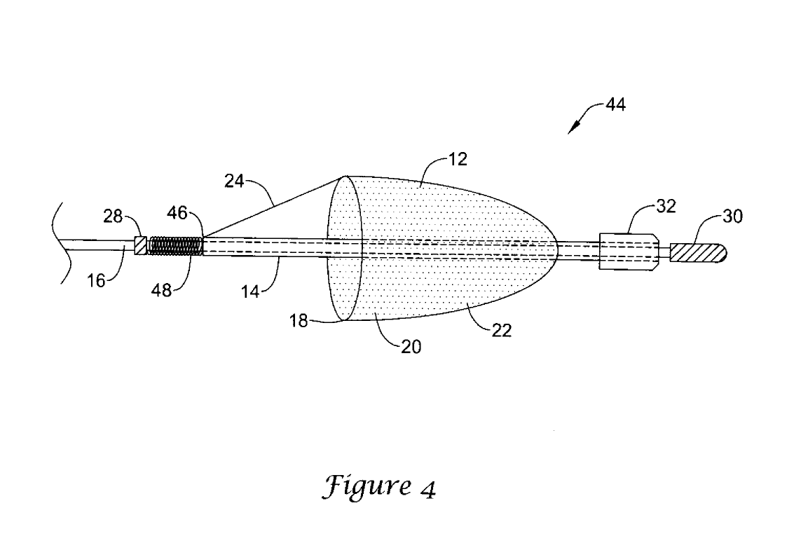

FIG. 4 provides an illustrative but non-limiting example of a filter assembly 44 that has many components in common with the filter assemblies described with respect to FIGS. 1, 2 and 3. In FIG. 4, spinner tube 14 has a proximal end 46. A spring 48 is disposed between proximal end 46 of spinner tube 14 and spinner tube stop 28. Much like compliant portion 42 of spinner tube 36 (FIG. 3), spring 48 may shorten or lengthen in response to axial movement of wire 16.

Spring 48 may be disposed about wire 16, and may be secured at either end to spinner tube 14 and spinner tube stop 28 in any suitable manner. Spring 48 may be a metallic or a non-metallic coil having a spring force constant. While not expressly illustrated, spring 48 may instead be an elastomeric cylinder. Spring 48 may include a polymeric sleeve to provide a smooth outer surface to ease advancement of wire 16 within a vasculature.

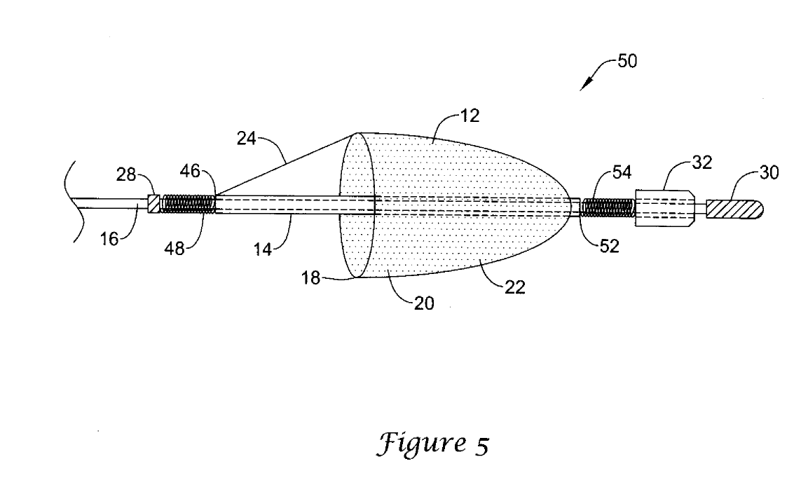

In FIG. 5, filter assembly 50 includes a second spring 54 that is disposed between a distal end 52 of spinner tube 14 and distal stop 32. Second spring 54 may be useful if, for example, distal stop 32 binds on wire 16. This may happen, for example, if filter assembly 50 is positioned within a tortured vasculature, and is substantially curved.

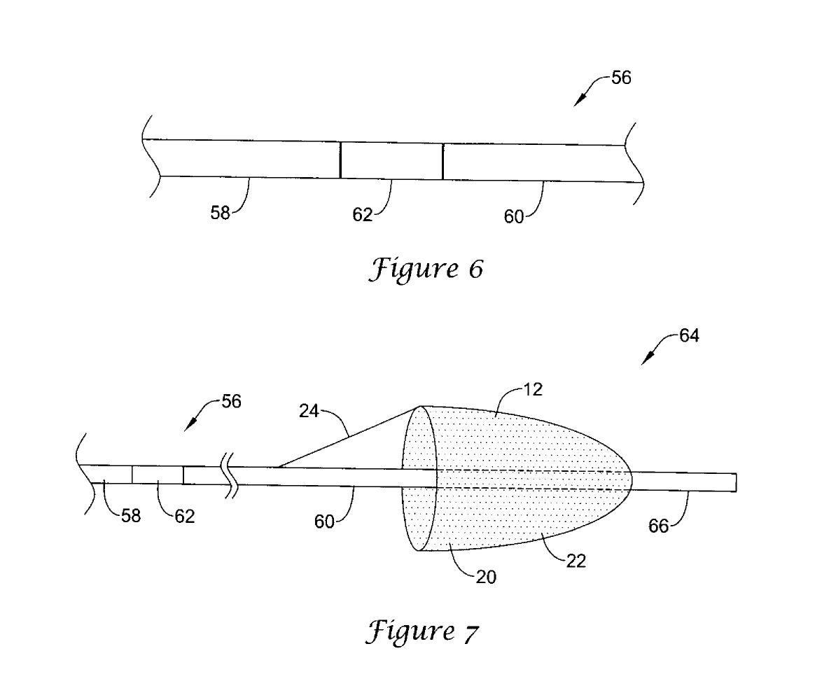

FIGS. 6 through 11 provide illustrative but non-limiting examples of telescoping wires. These telescoping wires may be used as guide wires, filter wires, or in any other application when it may be desirable to isolate a distal portion of the wire from movement in the proximal portion.

FIG. 6 illustrates a telescoping wire 56 that includes a proximal wire section 58, a distal wire section 60 and a joining section 62. Proximal wire section 58 may be solid or hollow, and may be formed of any suitable material. In some instances, proximal wire section 58 is solid, and is formed from or includes a metallic material such as stainless steel or a nickel/titanium alloy. Similarly, distal wire section 60 may be may be solid or hollow, and may be formed of any suitable material. In some instances, distal wire section 60 is solid, and is formed from or includes a metallic material such as stainless steel or a nickel/titanium alloy.

Joining section 62 may be configured to translate rotational movement from proximal wire section 58 to distal wire section 60, or vice versa, while permitting proximal wire section 58 to independently move axially relative to distal wire section 60, or vice versa. In some instances, as will be discussed in greater detail with respect to subsequent Figures, joining section 62 may be fixedly secured to proximal wire section 58 while distal wire section 60 may be slidingly disposed relative to joining section 62. Joining section 62 may be formed of any suitable metallic material such as stainless steel or a nickel/titanium alloy.

The relative position of joining section 62 may vary. For example, proximal wire section 58 and distal wire section 60 may be approximately equal in length, and joining section 62 may be disposed therebetween. In some cases, proximal wire section 58 may be either longer or shorter than distal wire section 60. However, for flexibility purposes, it may not be desirable for joining section 62 to be too close to a distal end (not illustrated in FIG. 6) of telescoping wire 56.

In some cases, in order to limit how close joining section 62 is to the distal end of telescoping wire 56, distal wire section 60 may be considered as being at least 5 percent of the overall length of telescoping wire 56. Distal wire section 60 may be at least about 10 percent, at least about 15 percent, at least about 20 percent, at least about 25 percent, or even more of the overall length of telescoping wire 56.

Another factor in determining the relative lengths of proximal wire section 58 and distal wire section 60 is that telescoping wire 56 may be deployed within a guide catheter (not shown). It may be desirable, therefore, to design the relative lengths of proximal wire section 58 and distal wire section 60 such that joining section 62 is contained within a portion of telescoping wire 56 that remains within a guide catheter. These relative lengths may be tailored, then, for a desired application.

FIG. 7 provides an illustrative but non-limiting example of a filter assembly 64, in which a filter 12 (as described previously) is secured to distal wire section 60 of telescoping wire 56, at or near a distal end 66 thereof. As noted above, joining section 62 may be configured to transmit rotational movement but exclude axial movement between proximal wire section 58 and distal wire section 60.

It will be appreciated that filter assembly 64, as seen in FIG. 7, may be advanced through a vasculature until a treatment site is reached. Filter 12 may then be deployed (as illustrated). One or more therapeutic devices may then be advanced over telescoping wire 56. In order to limit such activities from impacting the position of filter 12, it is noted that joining section 62 may be configured to permit proximal wire section 58 to be moved proximally a short distance. Then, any minor axial movement of proximal wire section 58 will not be transmitted to distal wire section 60.

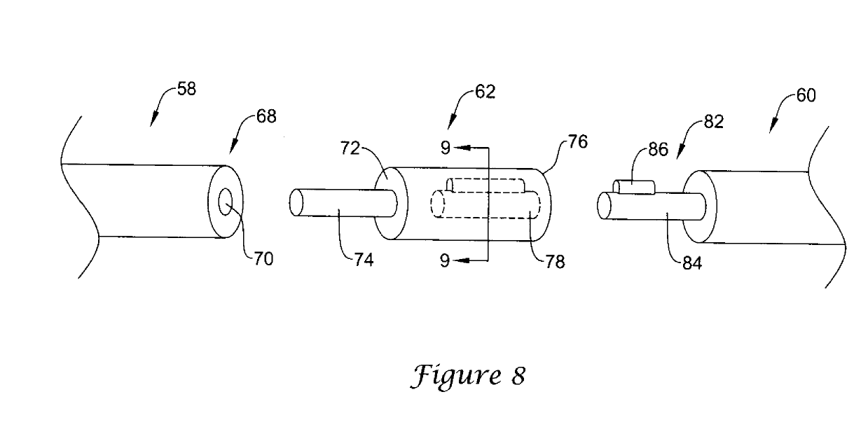

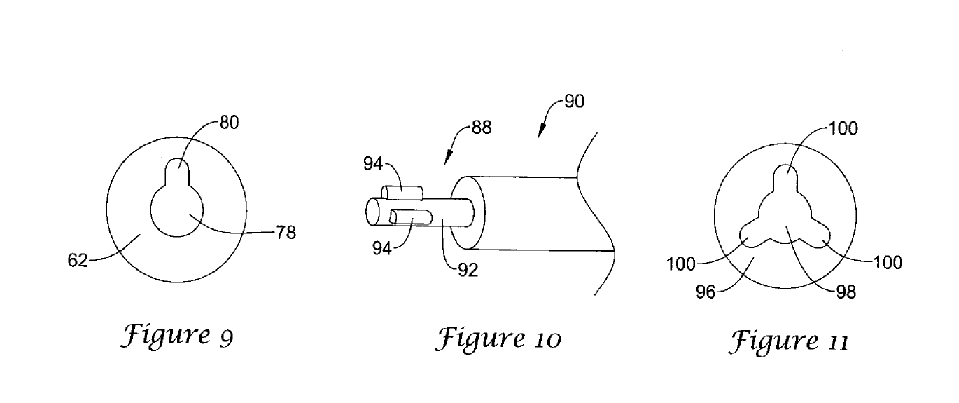

FIGS. 8 and 9 provide an illustrative but non-limiting example of how proximal wire section 58, distal wire section 60 and joining section 62 may interact in order to retain rotational travel between proximal wire section 58 and distal wire section 60 yet limit, when desired, relative axial movement therebetween. FIG. 8 is an exploded partial cross-sectional view of telescoping wire 56 while FIG. 9 is a cross-section taken through joining section 62.

In FIG. 8, a portion of proximal wire section 58 can be seen. Proximal wire section 58 includes a distal end 68 bearing a joining aperture 70. Similarly, joining section 62 includes a proximal end 72 bearing a joining extension 72. It can be seen that joining extension 72 may extend into joining aperture 70, thereby providing increased surface area for bonding, welding, or the like, and thereby provide a strong connection between proximal wire section 58 and joining section 62. While not illustrated, it is contemplated that a simple butt joint may be formed between distal end 68 of proximal wire section 58 and proximal end 72 of joining section 62.

It should be noted that while this discussion and the accompanying Figures describe joining section 62 being fixedly secured to proximal wire section 58 and moveably disposed about distal wire section 60, these may reversed. In other words, it is contemplated that proximal wire section 58 may be movably disposed while distal wire section 60 is fixedly secured to joining section 62.

Joining section 62 also includes a distal end 76 bearing a sliding aperture 78. Sliding aperture 78, as better seen in FIG. 9, includes a slot 80. Distal wire section 60 includes a proximal portion 82 including a narrowed segment 84 bearing a tab 86. Slot 80 disposed within sliding aperture 78 may be sized to slidingly accommodate tab 86. It can be seen in FIG. 87 that while sliding aperture 78 extends to the distal end 76 of joining section 62, slot 80 does not.

Thus, once telescoping wire 56 has been assembled, tab 86 may move axially back and forth within slot 80, yet tab 86 is held captive within slot 80. As a result, proximal wire section 58 may move axially without moving distal wire section 60, yet distal wire section 60 may not become completely detached from joining section 62.

Assembly may include any appropriate steps or techniques. In some cases, it is contemplated that the materials used in assembling telescoping wire 56 may be sufficiently resilient to permit narrowed segment 84, including tab 86, to be forcibly inserted into sliding aperture 78 and slot 80, yet once inserted, not permit removal of narrowed segment 84 from sliding aperture 78. Although not illustrated, tab 86 may include an angled leading edge to facilitate assembly. In some instances, narrowed segment 84 may be fully inserted into sliding aperture 78, and then welding or a similar technique may be used to close the distal end of slot 80. In some cases, shape memory materials may be used to facilitate assembly.

As illustrated, telescoping wire 56 includes a single slot 80 and a single, corresponding, tab 86. It will be recognized that any number of slots and tabs may be employed. FIG. 10 illustrates a proximal portion 88 of a distal wire section 90 including a narrowed segment 92. Narrowed segment 92 includes three tabs 94. Tabs 94 may be either equally or non-equally spaced. FIG. 11 is a diagrammatic cross-sectional view of a corresponding proximal wire section 96 that includes a sliding aperture 98 and three slots 100.

The filters and wires described herein may include a variety of different materials. These materials may include metals, metal alloys, polymers, metal-polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; other Co—Cr alloys; platinum enriched stainless steel; or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In addition, the devices described herein may also be doped with or otherwise include a radiopaque material. Radiopaque materials are understood to be materials capable of producing a relatively bright image on a fluoroscopy screen or another imaging technique during a medical procedure. This relatively bright image aids the user of filtering device in determining their location. Some examples of radiopaque materials can include, but are not limited to, gold, platinum, molybdenum, palladium, tantalum, tungsten or tungsten alloy, plastic material loaded with a radiopaque filler, and the like.

The invention should not be considered limited to the particular examples described above, but rather should be understood to cover all aspects of the invention as set out in the attached claims. Various modifications, equivalent processes, as well as numerous structures to which the invention can be applicable will be readily apparent to those of skill in the art upon review of the instant specification.

1. A filter assembly, comprising:
a filter wire having a proximal portion and a distal portion;
a tubular element having a proximal end and a distal end, said tubular element including a compliant portion between its distal and proximal ends and being rotatably disposed about the distal portion of the filter wire; and
a filter secured to the tubular element and located substantially between the proximal and distal ends thereof;
wherein axial movement of the filter wire is at least partially absorbed by the compliant portion of the tubular element.

2. The filter assembly of claim 1, wherein the tubular element has a length, and is adapted to change its length in response to axial movement of the filter wire.

3. The filter assembly of claim 1, wherein the compliant portion of the tubular element has a spring force constant.

4. The filter assembly of claim 3, wherein the tubular element includes a distal tubular portion and a proximal tubular portion, and the compliant portion having a spring force constant is disposed between the distal portion and the proximal portion and connected thereto.

5. The filter assembly of claim 4, wherein the compliant portion having a spring force constant comprises an accordion portion.

6. The filter assembly of claim 4, wherein the compliant portion having a spring force constant comprises an elastomeric portion.

7. A filter assembly, comprising:
a filter wire having a proximal portion and a distal portion, the distal portion including a distal end;
a tubular element having a proximal end and a distal end, said tubular element being rotatably disposed about the distal portion of the filter wire;
a filter secured to the tubular element and located substantially between the proximal and distal ends thereof;
a tubular element stop secured to the filter wire such that the tubular element is disposed distal the tubular element stop; and
an adaptable segment disposed between the tubular element stop and the tubular element proximal end, said adaptable segment being capable of changing length in response to axial movement of the filter wire.

8. The filter assembly of claim 7, wherein the tubular element has a length, and is adapted to change its length in response to axial movement of the filter wire.

9. The filter assembly of claim 7, wherein the adaptable segment comprises a spring.

10. The filter assembly of claim 7, wherein the tubular element further comprises a distal stop secured to the tubular element such that the distal stop is slidingly disposed about the filter wire.

11. The filter assembly of claim 10, further comprising a floppy tip secured to the distal end of the filter wire, the floppy tip limiting distal movement of the distal stop.

12. The filter assembly of claim 11, wherein the tubular element is permitted axial movement between the floppy tip and the tubular element stop.

13. The filter assembly of claim 10, further comprising a spring disposed between the distal stop and the distal end of the tubular element.

14. A telescoping wire, comprising:
a solid proximal wire section;
a solid distal wire section; and
a joining section disposed between the proximal wire section and the distal wire section, the joining section including a distal region and a proximal region;
wherein the joining section is configured to transmit rotational movement between the proximal wire section and the distal wire section yet permit the distal section to move axially relative to the proximal section.

15. The telescoping wire of claim 14, wherein the joining section is secured to the solid proximal wire section and the solid distal wire section is slidingly disposed relative to the joining section.

16. The telescoping wire of claim 14, wherein the solid proximal wire section includes a narrowed distal segment that extends into the proximal region of the joining section.

17. The telescoping wire of claim 16, wherein the solid distal wire section includes a narrowed proximal segment that extends into the distal region of the joining section.

18. The telescoping wire of claim 17, wherein the distal region of the joining section includes one or more axially aligned slots and the narrowed proximal segment of the solid distal wire section includes one or more axially aligned tabs that are complementary to the one or more axially aligned slots.

19. The telescoping wire of claim 18, wherein the one or more axially aligned tabs cooperate with the one or more axially aligned slots to permit relative axial movement but at least substantially limit relative rotational movement.

20. The telescoping wire of claim 14, further comprising an embolic filter secured to the solid distal wire section.
